(19) United States
(12) Patent Application Publication
(10) Pub. No.: US 2008/0319281 A1
(43) Pub. Date: Dec. 25, 2008

(54) Device for Detecting and Warning of Medical Condition

(75) Inventor: Ronaldus M. Aarts, Geldrop (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJLE PHILIPS ELECTRONICS, N.V., Eindhoven (NL)

(21) Appl. No.: 12/158,375

(22) PCT Filed: Dec. 19, 2006
(86) PCT No.: PCT/IB06/54952

§ 371 Date: Jun. 20, 2008

Related U.S. Application Data
(60) Provisional application No. 60/752,088, filed on Dec. 20, 2005.

(51) Int. Cl. A61B 5/00

(52) U.S. Cl. 600/301

(57) ABSTRACT

A portable device detects a medical condition, such as an epileptic seizure. The device is implemented on a wrist band, possibly together with a watch, or in a helmet. The device may use heart rate detection to identify characteristic patterns associated with epileptic seizure. The device optionally combines more than one measurement to eliminate false positives. In the case of epileptic seizures, heart rate related measurements may be combined with body motion related measurements to ensure greater accuracy.

The invention relates to the field of devices for detecting medical conditions, especially epilepsy.

Epilepsy is the most common neurological disorder after stroke, and affects almost 60 million people worldwide. Medications control seizures in only two thirds of those affected, and another 7%-8% are potentially curable by surgery. This leaves fully 25%, or 15 million people, whose seizures cannot be controlled by any available therapy. Over the past ten years, engineers and quantitative scientists have amassed evidence that seizures do not begin abruptly, as was previously thought, but develop over time, even hours before they cause clinical symptoms

Research has determined that heart rate measurements can predict epileptic seizures. Please see M. Zijlmans, D. Flanagan, and J. Gotman, “Heart rate changes and ECG abnormalities during epileptic seizures: prevalence and definition of an objective clinical sign”, Epilepsia, Vol. 43, No. 8, p. 847-854, 2002; and M. J. P. van Bussel, “Detection of epileptic seizures based on heart rate patterns”, MSc. report TU/e, Kempenhaeghe, Student number 0462628, Graduate professor J. Bergmans, April 2005.

It is an object of the invention to make prediction of medical phenomena, such as epileptic seizures, more accurate and more convenient.

Convenience can be achieved by creating a portable device, such as a wrist watch or helmet, which incorporates detection, processing and alarm functionality.

Accuracy can be achieved by implementing multiple detection functionalities and combining their outputs in a processor to cross check results and eliminate false positives.

Further objects and advantages will be apparent in the detailed description of the invention and the claims.

The invention will now be described by way of non-limiting example with respect to the drawing that includes:

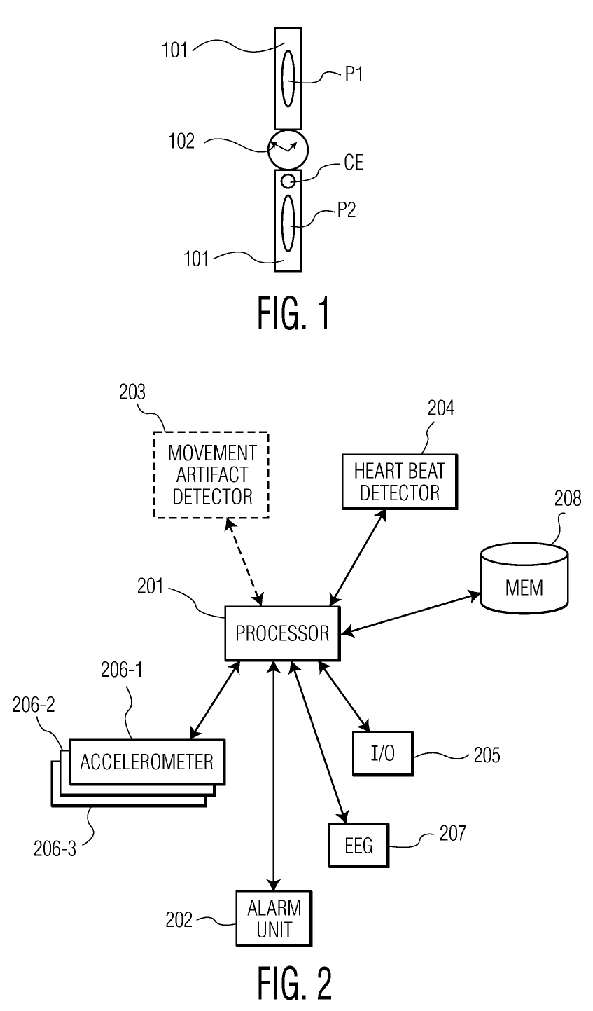

FIG. 1, which shows a wrist band borne device for detecting and/or predicting epileptic seizures;

FIG. 2, which is a schematic diagram of the device of FIG. 1;

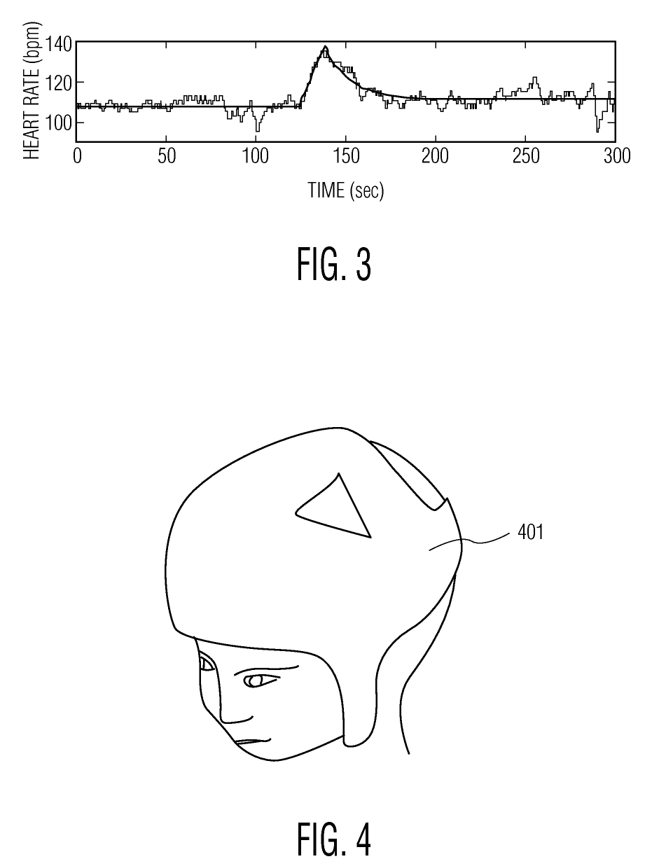

FIG. 3, which is a graph of heart rate against elapsed time showing a pattern characteristic of epileptic seizure;

FIG. 4, which shows an epilepsy helmet in which a device in accordance with the invention may be situated;

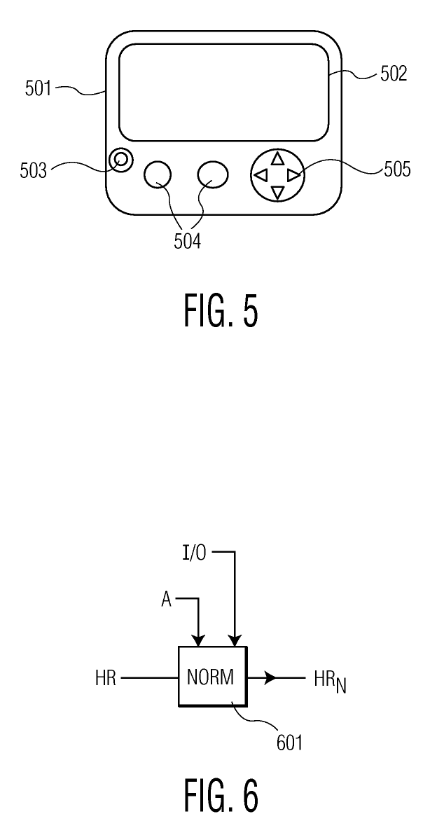

FIG. 5 shows a user interface device;

FIG. 6 is a schematic of a heart rate normalization unit;

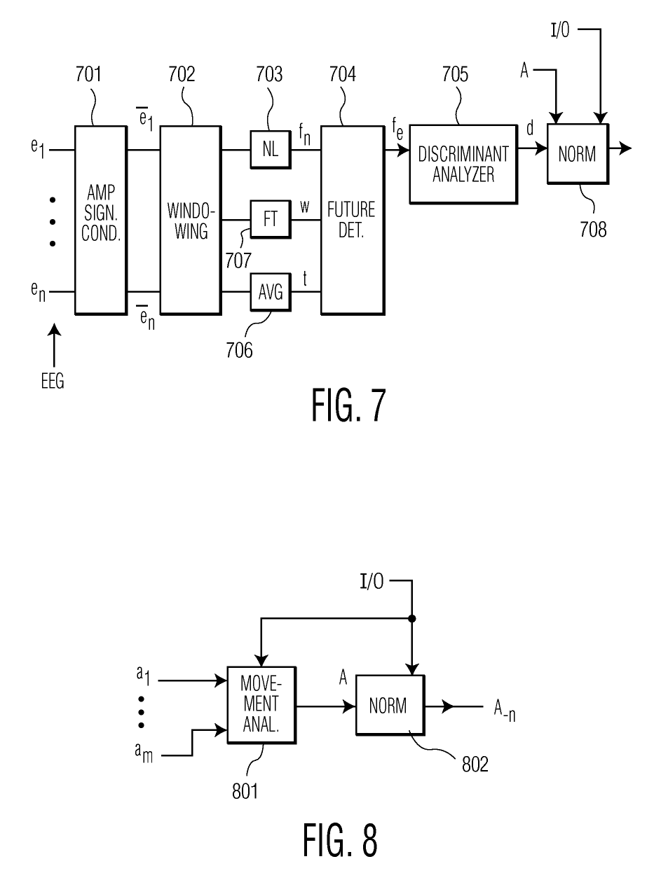

FIG. 7 is a schematic of an EEG analysis unit;

FIG. 8 is a schematic of an analysis unit for the accelerometers;

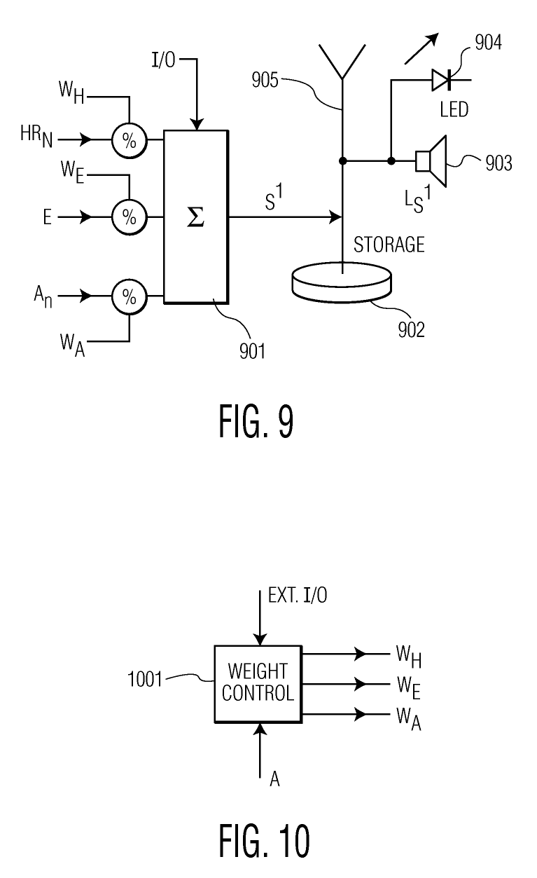

FIG. 9 is a schematic of an alarm generator; and

FIG. 10 is a schematic of a weighting control unit.

FIG. 1 shows a wrist band borne device for detecting and/or predicting a medical condition, such as an epileptic seizure. The device includes a wrist band 101 and an optional time piece 102. While it may be convenient to the patient for the device to tell time, like a wrist watch, such a time piece is not necessary to the functioning of the invention. The wrist band includes pads P1 and P2, which include sensor devices such as electrodes. More or less pads P1 . . . Pn may be used. The wrist band also includes a processing and display section CE.

Other types of portation modalities, such as head bands or chest bands, might be used to carry a device in accordance with the invention. Advantageously, epilepsy seizure detection equipment might be installed in an epilepsy helmet 401, as shown in FIG. 4.

FIG. 2 shows a schematic of sub-devices to be used in the preferred embodiment for detecting epileptic seizure. Processor 201 is for controlling the other units and for processing data signals from them. The processor interacts with at least one memory unit 208, which stores data and program code. The data may include seizure detection threshold or pattern information for use with the other devices. Preferably the memory can retain history information for periodic review by a health care professional who wishes to monitor seizure activity. History may be retained for long periods of time such as a month or a year, if infrequent medical review and/or download are expected. Alternatively, history may be retained for shorter periods of time, such as a day, if more frequent download and/or review are expected. The processor 201 is shown as being separate from the other devices, but some processing function may be distributed to local processors within the sensing devices.

A heart beat detector 204 is used to supply signals characterizing the heart beat of the wearer. Such a heart beat detector is discussed in the articles cited at the start of this application. This detector can detect heart rate, analogously to the device of U.S. Pat. No. 5,795,300, or it can be a more sophisticated EKG (electrocardiogram) type device that actually collects waveforms associated with heartbeat. An embodiment of the heart rate detector is shown in co-pending application Ser. No. ______ (ID 690694).

Heart rate detection alone may be used to detect seizure; however, since changes in heart beat type or heart rate can be caused by conditions other than epileptic seizure, other sensing devices are desirable to eliminate false positives. For instance, heart beat changes relating to seizure may in some cases be difficult to distinguish from heart beat changes associated with exercise or other body motions.

One other sensor device that may be desired is a movement artifact detector such as is shown at 203. Such a detector is disclosed in L. B. Wood & H. H. Asada, “Active Motion Artifact Reduction for Wearable Sensors Using Laguerre Expansion and Signal Separation,” Proceedings of the 2005 IEEE, Engineering in Medicine and Biology 27^{th} Annual Conference, Shanghai, China Sep. 1-4, 2005. This type of detector can correct for heart rate measurement errors that stem from movement of the device.

It may also be useful to include one or more accelerometers 206-1, 206-2, 206-3. Typically there will need to be more than one accelerometer to detect acceleration in multiple directions, for instance 3 to detect motion in 3 directions. The accelerometers may effect the movement artifact detection in conjunction with an appropriate processor, which may be at 201, or local to device or devices 206-1, 206-2, 206-3, thus rendering the separate device 203 unnecessary. The device 203 is shown in dotted lines to indicate that it is optional. In addition to detecting motion due to exercise, an accelerometer may be used to detect hectic body movements associated with seizure so that those can be used in conjunction with or even instead of heart rate detection.

Motion and position detection can be realized using a GPS device. Such a device can track patient movement and position. This may be useful with a patient who is free to leave a clinic or residential facility. There is a well developed art of tracking objects using GPS and other means. Such tracking is often used in movies, for instance, to support animation. Other types of devices may be used to gather motion or position data, such as velocity meters or position sensors.

Optionally, the device may include a connection to an EEG (electroencephalograph) unit 207, so that heart beat and motion information can be correlated with brain activity in determining whether a seizure is present or imminent. EEG data is considered the gold standard in detecting epileptic seizure and commercial devices and algorithms are available for analyzing EEG data for seizure detection. If the device in accordance with the invention is carried on a wrist strap, signals from the EEG unit 207 would have to be conveyed to the processor 201 from the head, preferably either wirelessly or via skin conduction, e.g. as discussed in U.S. Pat. No. 6,859,657 (PHB 34,280), incorporated herein by reference. If the device in accordance with the invention is implemented on a head band, cap, or helmet, the EEG unit might be incorporated into the device.

When a medical condition—such as a seizure—is detected, an alarm is desirable, and can be given by alarm unit 202. This alarm unit may be of any suitable sort. It may be give an audible or visible indication. An alarm indication may be sent wirelessly to a local or remote monitoring station, whence emergency personnel may be dispatched to deal with the situation. During the onset of a seizure, a caregiver can come to the patient to administer drugs or position the patient more safely.

The device further includes an input or input/output (I/O) facility 205. This facility might be wired, such as a socket for receiving an electrical or optical cable, or wireless, such as a radio frequency (RF) or infrared (IR) receiver or transceiver. Alternatively, the facility 205 may allow insertion of a memory medium of some sort to provide new data and or software. This facility allows the device to be reprogrammed with criteria or algorithms for detecting the medical condition. These updates may be applicable to any or all of the detection modalities 203, 204, 206-1, 206-2, 206-3, and/or 207 or to the processor 201. The updates may stem from ongoing medical research or from clinical observation of idiosyncratic signal patterns associated with a particular patient.

For instance, the van Bussel thesis, cited at the beginning of this application, includes the diagram shown in FIG. 3. This diagram graphs heart rate in beats per minute against seconds. Such data gives rise to a model for a seizure related tachycardia that includes a linear acceleration, a possible plateau and an exponential deceleration. If the exponential deceleration displays an undershoot, the event is called a seizure related bradycardia following a tachycardia. A pattern recognition algorithm in the processor 201 can look for this pattern. If further research at some future time reveals further information, new patterns or algorithms can be entered into the processor 201.

The field of artificial intelligence has identified several ways of integrating results from multiple sensing modalities. The article H. Witte, L. D. Iasemidis, and B. Litt, “Special Issue on Epileptic Seizure Prediction,” IEEE Transactions on Biomedical Engineering, pp. 537-539, 50 (5), May 2003 describes using a genetic algorithm to combine multiple EEG inputs to predict seizure. U.S. patent application Ser. No. 09/718,255 filed Nov. 22, 2000 (US 000293), incorporated herein by reference, discusses one type of multi-modal integration. PCT document WO0242242 is a counterpart of this application. The processor 201 can use an artificial intelligence technique such as those described in the above documents to combine the results from the various modalities 203, 204, 206-1, 206-2, 206-3, and 207. Correlation analysis might also be used. Alternatively, as discussed below, a mere sum of normalized and weighted signals may be used.

Combining results from several modalities reduces the likelihood of false positives. For instance, in the field of seizure detection, an accelerating heart rate pattern could potentially result from exercise and be confused with a seizure by a pattern recognition algorithm.

More broadly, a portable device—such as a wrist band borne device with multiple sensing modalities to eliminate false positives—can be used to detect other medical conditions, such as leg movement syndromes relating to sleep disorders or sleep walking. In these situation, heart rate combined with movement or acceleration indications could also indicate presence of the condition. Other types of sensing modalities might be used to detect other conditions.

Moreover, the individual devices within the unit may be used separately for other purposes. For instance, the outputs of a heart rate monitor or accelerometer may be useful to the patient who is engaging in athletic activities. It would be desirable for the device to offer the patient a choice of breaking out these individual outputs for purposes of the patient's choosing.

FIG. 5 shows a user interface device which may be used at 205. The device 501 may include a screen 502, a socket 503 for insertion of a cable or wire for inputting data, control buttons 504, and cursor control 505. This device may be situated at point CE on the wristband, along with the processor 201. Such an interface can also be installed on a helmet 401, preferably in a recessed or padded location. The screen 502 may be used to give the patient directions in how to use the device or to give an alarm indication warning of an impending seizure. The screen 502 may also give directions or other information to service or medical personnel who seek to read or update the device. Connector 503 may be connected to a keyboard or other data entry device or to a data processing device that transmits data or code. Data may also be entered manually via the control buttons 504 and cursor control 505. Other buttons or control devices may be used instead of those shown in FIG. 5, in accordance with design choice. The interface device may incorporate a loudspeaker in addition to or instead of the screen, for communication with a user. LED indicators may also be added or substituted.

In general, all of the electronics of the invention must be hardened or padded in such a way as to protect them during hectic limb movements due to seizure.

The circuitry of FIGS. 6-10 may be located in the sensors 203, 204, 206-1, 206-2, 206-4, and 207 or in the processor 201 or distributed between the sensors and the processor. Moreover, the functions shown in these figures may be implemented either in hardware or in software.

FIG. 6 is a schematic of heart rate normalization unit 601. This unit may be incorporated within the heart rate detector 204. Alternatively, it could be part of the processor 201. Normalization could be implemented in either hardware or software. The heart rate HR is normalized with activity level A coming from the signal analysis block in FIG. 8 and the thresholds given by the doctor via input I/O from 205 to yield a normalized heart rate HR_{N}. Normalization is done to allow outputs of different modalities to be added together. For instance, if heart rate varies between, for instance, 50 and 220, then the doctor can enter a heart rate such as 220 into the I/O device 205 so that the range becomes from 0 to 1. The other devices marked “norm” below similarly will make the ranges of their outputs between zero and one.

FIG. 7 is a schematic of an EEG analysis unit. Signals arriving from the EEG electrodes e₁ . . . eₙ are amplified and processed by block signal conditioning unit 701. This conditioning includes filtering and noise reduction. The outputs of the conditioning unit are e̅₁̅ . . . e̅ₙ̅. These are fed to the windowing block 702, which has three outputs. Windowing is used to choose the length of data to be used. More about windowing can be found in the book A. v. Oppenheim & R. W. Schaffer, Discrete-Time Signal Processing (Prentice Hall 1989) for instance at pp 444-462. The first output of box 702 goes to a nonlinear processing block 703 yielding an output fₙ. Another output of the windowing block 702 goes to a Fourier transform block 707, yielding an output Co. A third output of the windowing block 702 goes to an averaging unit 706, yielding an output t. The three outputs fₙ, ω, and t are then fed to a block feature detection unit 704 which outputs features called fₑ, which in turn are fed to a block discriminant analyzer 705. The block discriminant analyzer 705 supplies an output d which is normalized at 708, analogously to the normalization at block 601 in FIG. 6. Block feature detection and discriminant analysis per units 703-705 are further described in N. Paivinen, “Epileptic Seizure Detection: a Non-linear Viewpoint,” Computer Methods and Programs in Biomedicine (2005) 79, 151-159.

FIG. 8 is a schematic of an analysis unit for use with the accelerometers. Inputs a₁ . . . aₘ from the accelerometers are fed to movement analysis unit 801. In this unit, the inputs are filtered to eliminate signals not consistent with human movement, under control of any input received. The output A from the unit 801 is fed to a normalization unit 802 and normalized in view of inputs from the i/o device 205. These inputs will be from a doctor or other device operator and will include data relevant to the individual patient, such as sex, weight and age, which will help determine normal ranges of movement for that person.

FIG. 9 is a schematic of an alarm generator. Normalized inputs HR_{N}, E, and Aₙ are received from the units of FIGS. 6, 7, and 8, respectively. To these inputs, are applied respective weights, w_{H}, w_{E}, and w_{A}. Initially the weights can be set at 1, but later they can be changed in response to inputs, to emphasize or deemphasize a parameter. The connection from the line i/o to the lines w_{H}, w_{E}, and w_{A} is not shown to simplify the diagram. The weighted inputs are then summed at 901 to yield a final signal S, which can be made audible by a buzzer or loudspeaker 903, or a visible by a device such as LED 904 or screen 502. Alternatively, output can be wireless 905 and transmitted to a caregiver.

FIG. 10 shows a weighting control unit 1001 that takes inputs A from the unit of FIG. 8 and i/o from the unit 205 and generates therefrom w_{H}, w_{E}, and w_{A}. Unit 1001 can be a look up table.

From reading the present disclosure, other modifications will be apparent to persons skilled in the art. Such modifications may involve other features which are already known in the design, manufacture and use of medical devices and which may be used instead of or in addition to features already described herein. Although claims have been formulated in this application to particular combinations of features, it should be understood that the scope of the disclosure of the present application also includes any novel feature or novel combination of features disclosed herein either explicitly or implicitly or any generalization thereof, whether or not it mitigates any or all of the same technical problems as does the present invention. The applicants hereby give notice that new claims may be formulated to such features during the prosecution of the present application or any further application derived therefrom.

The word “comprising”, “comprise”, or “comprises” as used herein should not be viewed as excluding additional elements. The singular article “a” or “an” as used herein should not be viewed as excluding a plurality of elements. The word “or” should be construed as an inclusive or, in other words as “and/or”.

1. A portable device for predicting epileptic seizures comprising:
at least one sensor (203, 204, 206-1, 206-2, 206-3, 207) for sensing a physical phenomenon in a patient's body, which phenomenon is known to be able to predict epileptic seizures, and for supplying signals characteristic of that phenomenon;
at least one processor (201), coupled with the sensor (203, 204, 206-1, 206-2, 206-3, 207), for performing operations, the operations comprising processing the signals to determine whether they meet at least one criterion characteristic of epileptic seizures;
at least one output device (205, 501, 903, 904, 905) adapted to supply an alarm indication, when the criterion is met; and
at least one fastening apparatus for affixing the device to a patient's body.

2. The device of claim 1, wherein the fastening apparatus comprises a wrist band (101) carrying at least the processor (CE) and the output device (CE).

3. The device of claim 1, wherein the means for affixing comprises headgear (401) carrying the sensor, the processor and the output device.

4. The device of claim 1, wherein
the phenomenon comprises a heart beat pattern (FIG. 3);
the at least one criterion comprises at least one stored heartbeat pattern known to be predictive of epileptic seizures; and
the processing means uses an artificial intelligence algorithm to determine whether the signals match the stored pattern.

5. The device of claim 4, wherein
the sensor further comprises at least one motion detection device (204, 206-1, 206-2, 206-3) suitable for detecting movement artifacts, acceleration, or both;
the signals supplied by the sensor comprise signals relating to heartbeat pattern and one or more of movement artifacts and acceleration in at least one direction;
the means for processing analyzes the signals in accordance with at least two criteria, one for each of the types of signals supplied by the sensors to create a combined analysis result; and
the alarm indication is supplied or not supplied responsive to the combined result.

6. The device of claim 1, wherein
the sensor comprises a plurality of sensors including a heart beat detector 204, at least three accelerometers (206-1, 206-2, 206-3) for detecting motion in three dimensions, and an EEG device (207);
the criterion comprises a plurality of criteria, including:
at least one heart beat pattern associated with epileptic seizure;
at least one first motion criterion designed to distinguish normal motion from seizure motion;
at least one second motion criterion designed to correct heart beat measurements for movement artifacts; and
at least one EEG criterion associated with epileptic seizure; and
the operations comprise considering signals from the plurality of sensors in view of the plurality of criteria in order to determine the presence or absence of epileptic seizure.

7. The device of claim 1 wherein the processing device is adapted to use the signals from the sensor for a second purpose separate from detecting seizures.

8. The device of claim 6 wherein processing comprises weighting (w_{H}, w_{E}, and w_{A}) and adding (901) normalized signals from the sensors.

9. The device of claim 1, wherein the sensor comprises at least one movement artifact detector (203) and the operations include correcting signals from at least one other detector responsive to at least one movement artifact.

10. The device of claim 1, wherein the sensor comprises at least one accelerometer (206-1, 206-2, 206-3).

11. The device of claim 1, further comprising an input (205) for receiving data and/or programming updates.

12. The device of claim 1, wherein the alarm indication is provided locally (502, 903, 904) to the device.

13. The device of claim 1, wherein the alarm indication is transmitted wirelessly (905) to a monitoring station.

14. A method for detecting seizures comprising performing the following operations in at least one portable electronic device affixed to a patient's body:
sensing a physical phenomenon in the patient's body, which phenomenon is known to be able to predict epileptic seizures, and for supplying signals characteristic of that phenomenon;
performing operations in at least one processor, the operations comprising processing the signals to determine whether they meet at least one criterion characteristic of epileptic seizures; and
supplying an alarm indication when the criterion is met.

15. The method of claim 14, wherein the phenomenon comprises heart beat and the criterion comprises a known heart beat pattern.

16. The method of claim 14, wherein the phenomenon comprises motion and the criterion comprises a known motion pattern.

17. The method of claim 14, wherein
the phenomenon comprises a plurality of phenomena including heart beat and motion;
the criterion comprises a plurality of criteria including:
a known heart beat pattern associated with seizure; and
a known motion pattern associated with seizure; and
the processing comprises correcting the signals for movement artifacts and comparing the signals with the criteria.

18. A medium readable by at least one data processing device and comprising code for causing the device to implement the method of claim 14.

19. A device for detecting a medical condition, comprising:
a portable apparatus (101, 501) suitable for attachment to and wearing on a patient's body;
at least one sensor (203, 204, 206-1, 206-2, 206-3, 207) coupled with the apparatus and adapted to measure at least first and second physical properties of the patient's body and to supply signals indicative of those properties; and
at least one processor (201) disposed within the apparatus and adapted to perform operations, the operations comprising:
analyzing the signals using at least first and second criteria relating to the first and second properties, respectively, to determine whether both the first and second properties taken together indicate the medical condition; and
if the medical condition is indicated, supplying an alarm indication.

20. The device of claim 19, wherein the at least one sensor comprises at least a single sensor (206-1) making a single measurement which is used in determining at least two physical properties of the patient's body.
